(19) United States
(12) Patent Application Publication
(10) Pub. No.: US 2014/0322720 A1
(43) Pub. Date: Oct. 30, 2014

(54) COMPOSITION FOR DIAGNOSING, TREATING, AND PREVENTING AGE-RELATED MACULAR DEGENERATION AND METHOD FOR DIAGNOSING AGE-RELATED MACULAR DEGENERATION

(71) Applicant: KONKUK UNIVERSITY INDUSTRIAL COOPERATION CORP., Seoul (KR)

(72) Inventors: Hyewon CHUNG, Seoul (KR); Hyung-Soon PARK, Seoul (KR); Gum-Yong KANG, Seoul (KR)

(73) Assignee: KONKUK UNIVERSITY INDUSTRIAL COOPERATION CORP., Seoul (KR)

(21) Appl. No.: 14/096,647

(22) Filed: Dec. 4, 2013

(30) Foreign Application Priority Data
Apr. 26, 2013 (KR) 10-2013-0046993

(51) Int. Cl. G01N 33/68

(52) U.S. Cl. CPC G01N 33/6893
USPC 435/7.1; 436/501; 530/350; 530/357; 530/399

(57) ABSTRACT

There is provided a composition for diagnosing, treating, and preventing an age-related macular degeneration and a method for diagnosing an age-related macular degeneration.

### CROSS-REFERENCE TO RELATED APPLICATION(S)

This application claims the priority of Korean Patent Application No. 10-2013-0046993 filed on Apr. 26, 2013, the entire contents of which is incorporated herein by reference.

### BACKGROUND OF THE INVENTION

1. Field of the Invention

The present invention relates to a composition for diagnosing, treating, and preventing an age-related macular degeneration and a method for diagnosing an age-related macular degeneration.

2. Description of the Related Art

As well as Korea, the average length of life in the entire world is over 80 years, and we are living in the ageing era. Particularly, in Korea, the ageing is rapidly in progress due to a dramatic rise of the average length of life, birth rate drop, and the like.

It is very important to treat diseases caused by aging and external environment in order to maintain healthy life for the longevity. The most common diseases caused by the aging are a disease related to eyes. For an ophthalmologic disease that was meant to be a disease by the aging, the age of onset is declining, and also the attack rate is rising. For an age-related macular degeneration (AMD) that is the first disease of the diseases leading to blindness among adults order than 50 in the west, the attack rate is rapidly rising in Korea and the prevalence rate is also high.

An age-related macular degeneration (AMD) is a disease that for adults order than 50, retina, retinal pigment epithelium, and choriocapillaris are degenerated and sink into atrophy, progressively and multifactorially. In the west, the AMD is an important disease that takes first place among the diseases leading to blindness among adults order than 50, surpassing diabetic retinopathy and glaucoma. Latest estimates shows more than eight million people have a nonvasculitic or dry AMD that is more common type of the AMD in the United States, and more than three million people are losing their sight every year due to the AMD. In Korea, AMD incidence is rapidly rising due to westernization of dietary life, an environment factor, and a prolonged average life span. Currently, it is estimated that a quarter-million people, which is about 5% of five million people order than 65, suffer from the AMD. It is possible that such a geriatric illness leads to an economical problem such as burden of a medical expenses and also great social problem such as quality-of life decline. Therefore, a study on an early diagnosis and effective therapy of the AMD is urgently needed.

However, an etiopathogenic mechanism of an AMD is multifactorial and partially known. It is reported that in addition to a genetic factor, the AMD has relevance to an accumulation of a harmful environment such as environmental pollution, (excessive) ray exposure, and oxidation stress, but the detail mechanism is not completely known.

Currently, the treatment present condition of an AMD patient is as follows.

A therapy of choroidal neovascularization (CNV) that is a wet AMD is largely divided into a destructive therapy (macular grid laser treatment, a photodynamic therapy, and a surgery operation) and a pathway-based therapy (an anti-vascular endothelial growth factor therapy).

However, up to now, the therapies disclosed above only delay a serious vision loss or slightly improve a sight, but do not treat basically. Especially, the therapies only treat a wet AMD or neovascular AMD, but not treat a nonvascular AMD (a dry AMD and geographic atrophy), from which 70 to 80% of the AMD patients suffer.

A repeatable intravitreally-injection of an anti-VEGF is the only therapy, in which the injection has proven effect on treating a wet AMD now. The injection is a therapy targeting a vascular endothelial growth factor (VEGF) that is an angiogenic cytokine of choroidal neovascularization (CNV) of the wet AMD. However, a restricted visual improvement is proven only in ⅓ among the patients treated with an anti-VEGF therapy, and a legal blindness condition progresses in ⅙ among the patients treated with the therapy. In addition, there is a problem of stability such as retinal toxicity caused by continuously suppressing VEGF present in a normal retina. The therapy disclosed above should be repeatedly performed through an injection because of an intravitreal short half-life of a drug, and complications possibility such as a vitreous hemorrhage, retinal detachment and endophthalmitis increases according to the repeatable injection. Since the VEGF play a role as a strong vasodilator, the VEGF maintains a blood circulation and atony of the coronary arteries of heart. Since the aged patients with the AMD have high risk of occurring cardiovascular disorders, there is also a risk of occurring serious side effects. Accordingly, it is needed that an AMD-specific molecular or a protein marker is found and substances targeting the AMD-specific molecular or protein marker are developed.

### CITED DOCUMENTS

Korean Patent Publication No. 10-2012-0129023 (Nov. 28, 2012)
Korean Patent Publication No. 10-2009-0029259 (Mar. 20, 2009)

### SUMMARY OF THE INVENTION

The present invention is designed to solve the above-mentioned problems and by the above needs, and an aspect of the present invention provides a protein biomarker for diagnosing and treating an age-related macular degeneration.

According to an aspect of the present invention, there is provided a composition for diagnosing an age-related macular degeneration, in which the composition includes one or more proteins selected from the group consisting of desmoplakin-I represented by a gene information number (GI No.) 58530840; actin represented by GI No. 213688375; myosin 9 represented by GI No. 12667788; Hsp70 represented by GI No. 194248072; Hspβ-1 represented by GI No. 4504517; cytokeratin 8 represented by GI No. 4504919; cytokeratin 9 represented by GI No. 55956899; cytokeratin 14 represented by GI No. 15431310; vitronectin represented by GI No. 88853069; PSMD1 (26S proteasome non-ATPase subunit 1) represented by GI No. 25777600; cathepsin D represented by GI No. 4503143; a retinol-binding protein 4 represented by GI No. 55743122; and WIF 1 (Wnt inhibitory factor 1) represented by GI No. 111125011.

According to another aspect of the present invention, there is provided a composition for diagnosing an age-related macular degeneration, in which the composition includes a protein consisting of desmoplakin-I represented by a gene information number (GI No.) 58530840; actin represented by GI No. 213688375; myosin 9 represented by GI No. 12667788; Hsp70 represented by GI No. 194248072; Hspβ-1 represented by GI No. 4504517; cytokeratin 8 represented by GI No. 4504919; cytokeratin 9 represented by GI No. 55956899; cytokeratin 14 represented by GI No. 15431310; vitronectin represented by GI No. 88853069; PSMD1 (26S proteasome non-ATPase subunit 1) represented by GI No. 25777600; cathepsin D represented by GI No. 4503143; a retinol-binding protein 4 represented by GI No. 55743122; and WIF1 (Wnt inhibitory factor 1) represented by GI No. 111125011.

According to still another aspect of the present invention, there is provided a method for obtaining information about an age-related macular degeneration from a subject, in which the method includes (a) measuring one or more biomarkers in a biological sample obtained from the subject, in which the one or more biomarkers are selected from the protein compositions of the present invention disclosed above; and (b) correlating the sample with the age-related macular degeneration when measured value or values is/are higher than that of a normal person.

According to an embodiment of the present invention, it is preferable that for the method, when expression of the protein biomarker in the sample is at least 1.5 time or higher than the expression for the normal person, the sample is correlated to the age-related macular degeneration. However, the present invention is not limited thereto. In other words, in the case of proteins that are strongly presumed to be related to a pathological physiology of the age-related macular degeneration, the proteins are considered for selecting proteins for a final quantitative analysis, even if expressions of the proteins are not increased by 1.5 times or more.

Further, the present invention provides a composition for predicting an anti-VEGF medicine reaction for treating an age-related macular degeneration, in which the composition includes one or more proteins selected from the group consisting of Hspβ-1 represented by GI No. 4504517; and cytokeratin 8 represented by GI No. 4504919.

The present invention provides a kit for diagnosing a protein biomarker having at least one peptide selected from the group consisting of peptides set forth in SEQ ID NOS. 1 to 20 as an effective component.

In the present invention, an immunogenic fragment means a fragment of a biomarker protein having one or more epitopes capable of being recognized by an antibody to a biomarker protein of the present invention.

In order to achieve other purposes of the present invention, there is a provided a diagnostic agent for diagnosing an age-related macular degeneration, in which the diagnostic agent includes an antibody specifically binding the biomarker protein of the present invention or immunogenic fragment thereof as an effective component.

The antibody of the present invention may be a polyclonal antibody, or it is more preferable to use a monoclonal antibody.

The polyclonal antibody may be prepared by injecting a biomarker protein or fragments thereof as an immunogen to an external host according to the method known by a person skilled in the related art. Examples of the external host include a mammal such as a mouse, a rat, a sheep, and a rabbit. The immunogen is injected intramuscularly, abdominally, or subcutaneously. Generally, the immunogen is injected along with adjuvant to increase antigenicity. Blood is regularly collected from the external host, thereby collecting serum exhibiting improved titer or specificity to an antigen, or isolating and purifying an antibody from the blood.

The monoclonal antibody may be prepared by a technology [Koeher and Milstein 1975, Nature, 256:495)] of generating a cell line immortalized by the fusion that is known by a person skilled in the related art. A brief explanation of the generating method is as follows. First, 20 μg of a pure protein was obtained and then immunized to a Balb/C mouse, or a peptide was synthesized and bound to bovine serum albumin, and then immunized to the mouse. Since then, antigen-producing lymphocyte was isolated from the mouse, and then fused with myeloma of human or a mouse to generate immortalized hybridoma. Using an ELISA method, hybridoma that produces the desired monoclonal antibody was only selected and then proliferated, and then the monoclonal antibody was isolated and purified from the culture.

In order to achieve another purpose of the present invention, there is provided a method of obtaining information about an age-related macular degeneration of a subject, in which the method includes detecting in the presence of the biomarker protein of the present invention or immunogenic fragments thereof from a body fluid of the subject.

In the method of the present invention, using human blood, the detecting in the presence of a biomarker protein or immunogenic fragments thereof from the liver blood solution of a subject is directly performed by using a two-dimensional (2-D) electrophoresis, or the detecting in the presence of a biomarker protein or immunogenic fragments thereof is indirectly performed through an antigen-antibody reaction performed by contacting the blood with the antibody of the present invention.

Examples of an immunoassay method that is now widely known as the antigen-antibody reaction include an enzyme-linked immunosorbent assay (ELISA, Coated tube), a magnetic particle method, in which an antibody-binding magnetic particle is bound to a tube, and then an antigen-tracer and non-degradable contaminant are competitively reacted each other to induce an enzyme reaction, thereby quantifying, and a latex particle method using an antibody-binding latex particle.

In order to achieve still another object of the present invention, there is provided a kit for diagnosing an age-related macular degeneration, in which the kit includes an antibody specifically binding to a biomarker protein of the present invention or immunogenic fragments thereof as an effective component.

The diagnosing kit of the present invention is prepared by the preparing method that is known by a person skilled in the related art, and typically includes an antibody, a buffer, a stabilizer, an inert protein in a freeze-drying type. The antibody may be labeled with radioisotope, fluorogen, enzyme, and the like.

The monoclonal antibody of the present invention may be variously used for an immunoassay kit (ELISA, antibody coated tube test, lateral-flow test, potable biosensor), and may be also used for developing a protein chip having various spectrums for detecting an age-related macular degeneration through a development of an antibody exhibiting higher specificity and sensitivity.

In order to achieve another object of the present invention, there is provided a composition for treating and preventing an age-related macular degeneration, in which the composition includes a protein selected from the group increasing and decreasing an aqueous humor of a patient with an age-related macular degeneration as an effective component.

The pharmaceutical composition of the present invention may be prepared in a formulation, such as parenteral injections by mixing with a pharmaceutically acceptable carrier, excipient, diluents, and the like according to the method that is known in a pharmaceutical field, and then administered intravenously. A dose of the composition according to the present invention may be properly selected according to an age, sex, conditions, and disease symptom of a patient and the dose may be administered in an amount of 0.001 to 100 mg per a day based on an adult standard.

### BRIEF DESCRIPTION OF THE DRAWINGS

The above and other aspects, features and other advantages of the present invention will be more clearly understood from the following detailed description taken in conjunction with the accompanying drawings, in which:

FIG. 1. Overall strategy for the experimental procedures. (A) 1^{st} stage: Whole-proteome profiling of aqueous humor (AH) samples; (B) 2^{nd} stage: Whole-proteome profiling of conditioned medium (CM) from ARPE-19 cells; (C) 3^{rd} stage: Whole-proteome profiling of exosomal proteins in AH and CM; (D) 4^{th} stage: Liquid chromatography multiple reaction monitoring (LC-MRM);

FIG. 2. Venn diagram of the identified whole proteins from the AH of patients (patients 1 and 2) (A); the CM of ARPE-19 cells (control culture and culture exposed to 400 μM paraquat for 24 h) (B); exosomes from the AH of 9 pooled patients and 9 control subjects (C); and exosomes from the CM of ARPE-19 cells (control culture and culture exposed to 400 μM paraquat for 24 h) (D). Western blot analysis of cathepsin D and desmoplakin in the AH of a patient showed increased expression of these proteins compared to a control patient (E). The distribution and classification of proteins from the AH, the CM of ARPE19-cells, exosomes from the AH, and exosomes from the CM of ARPE-19 cells (F-H). The cellular distribution (F), molecular function (G), and biological process (H) profiles of the identified proteins are shown;

FIG. 3. Ultra-structural and biochemical characterization of the exosomes isolated from the AH of patients and CM of ARPE-19 cells. (A) A representative negative staining electron micrograph of exosomes released from the AH of patients (left) and the CM of ARPE-19 cells exposed to 400 μM paraquat for 24 h (right) (bar, 100 nm). (B) Western blot analysis of exosomes using antibodies recognizing the known exosomal marker CD63. The same amounts (15 μg) of cell lysates (from the ARPE-19 cells that produced the CM), exosomes from the CM and exosomes from the AH of patients were loaded on the same gel (con: control culture; para: culture exposed to 400 μM paraquat for 24 h). The expression of CD63 was increased in the exosomes from the AH of patients and CM exposed to 400 μM paraquat for 24 h compared to CM from control cultures. Cathepsin D was increased in cell lysates exposed to oxidative stress compared to controls as well as in the exosomes isolated from the AH of patients and in the CM from oxidatively stressed ARPE-19 cells compared to exosomes from control CM. Cathepins D* was found in AH profiling, and CM from ARPE-19 cells. (C) Fluorescent confocal photomicrographs of ARPE-19 cells immunostained for CD63. Compared to controls, ARPE-19 cells exposed to 400 μM paraquat for 24 h showed an increased globular pattern of CD63 staining (scale bar, 10 μm). For nuclear counterstaining, TO-PRO-3 (blue) was used. (D) Western blot analysis for further validation of several exosomal proteins using antibodies against Hsp70, cytokeratin 8, and 26S proteasome non-ATPase subunit 1 (PSMD1) and Tsg101. Hsp70 and cytokeratin 8 were found in AH profiling and CM from ARPE-19 cells. PSMD 1 was found in AH profiling;

FIG. 4. (A) LC-MRM analysis of 12 selected proteins in 15 AH sets (C: control, P: patient before treatment with ranibizumab, T: patient one month after treatment with ranibizumab). The relative abundances of the 12 proteins were elevated in the AH samples from patients before and one month after treatment compared to those from the control group as determined by t-test analysis (y-axis: Fold change compared to controls). (B) ROC curves of 12 selected proteins. Randomly chosen transitions from each protein were used in the analyses. The area under the curve (AUC) at a 95% confidence level is shown; and

FIG. 5 is a diagram illustrating WIF-1 expression (Western blot analysis) of aqueous humor of patients with an age-related macular degeneration, in which a WIF-1 protein expression was investigated before and after treatment with Lucentis, compared to age and sex-matched control subjects. As a result, in total 6 sample sets, it could be confirmed that a WIF-1 protein expression was clearly increased as compared with the control group.

### DETAILED DESCRIPTION OF THE PREFERRED EMBODIMENT

Exemplary embodiments of the present invention will now be described in detail with reference to the accompanying drawings.

Hereinafter, the present invention will be described.

To uncover novel secretory biomarkers related to the pathogenesis of AMD, we adopted an integrated approach to compare the proteins identified in the whole-proteomic profiling of the aqueous humor (AH) of patients with neovascular AMD, the conditioned medium (CM) of ARPE-19 cells, and the exosomes derived from the AH of patients and CM.

Label-free, semi-quantitative, LC-ESI-MS/MS-based proteomic profiling was performed on the AH of patients, the CM of ARPE-19 cells, and the exosomes derived from both samples. In addition, liquid chromatography multiple reaction monitoring (LC-MRM) analysis was performed on the AH from patients and control subjects. Our LC-MRM analyses revealed that heat shock protein 70, cytokeratin 8, vitronectin, the 26S proteasome non-ATPase subunit 1, cathepsin D, retinol-binding protein 4 and 5 other proteins were upregulated in the AH of 15 patients, and that the levels of these proteins decreased after the intravitreal injection of ranibizumab. The present study has identified potential biomarkers and therapeutic targets for AMD treatment, such as proteins related to molecular chaperones, the autophago-lysosomal pathway, the proteasome-ubiquitin pathway, and epithelial-mesenchymal transition, and demonstrated a novel and effective approach to identifying AMD-associated proteins that are secreted by RPE cells in vivo in the form of exosomes.

Hereinafter, the present invention will be described in detail.

Overall Strategy for Experimental Procedures

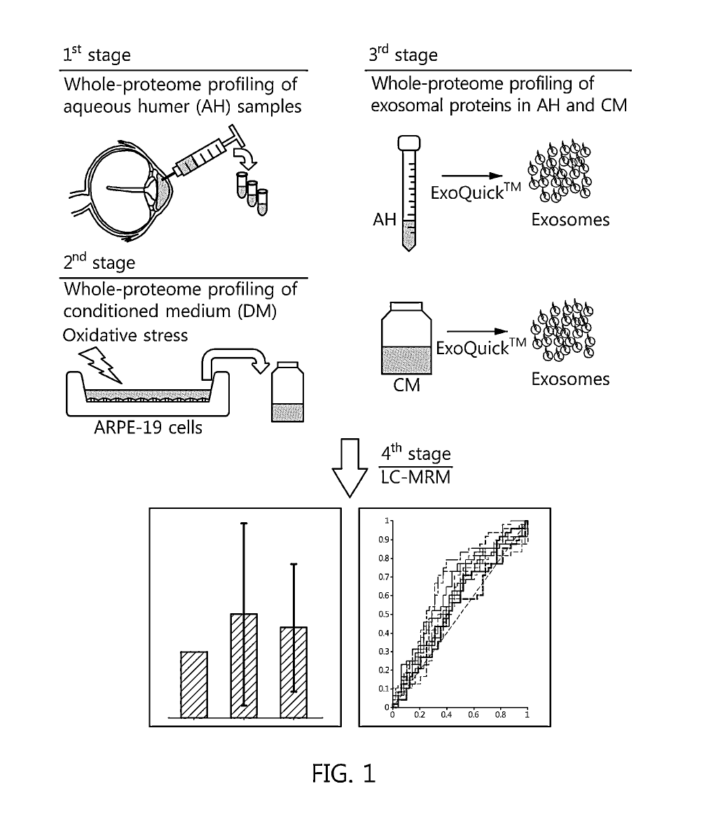

The present studies were carried out in four stages, as depicted in FIG. 1. In the first stage, we profiled the changes in the whole proteomes of the AH from two AMD patients and their controls by LC-ESI-MS/MS. We selected proteins that were upregulated in patients compared to controls for further analysis. In stage 2, we profiled the whole secretome, i.e., the CM, from oxidatively stressed ARPE-19 cells and compared these data with those generated in stage 1. In stage 3, we characterized the exosomal proteins in the AH of patients and in the CM of oxidatively stressed ARPE-19 cells. In stage 4, we determined the transitions for LC-MRM runs and performed LC-MRM analysis of the 12 proteins that were found in more than two proteomic profiles.

Proteomic Profiling of the AH from Two Patients with Neovascular AMD by LC-ESI-MS/MS and Biological Pathway Analysis of the Proteome

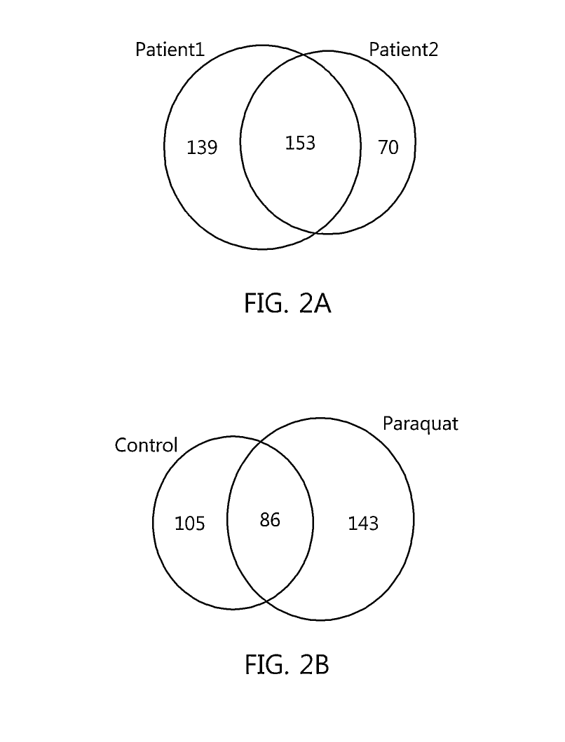

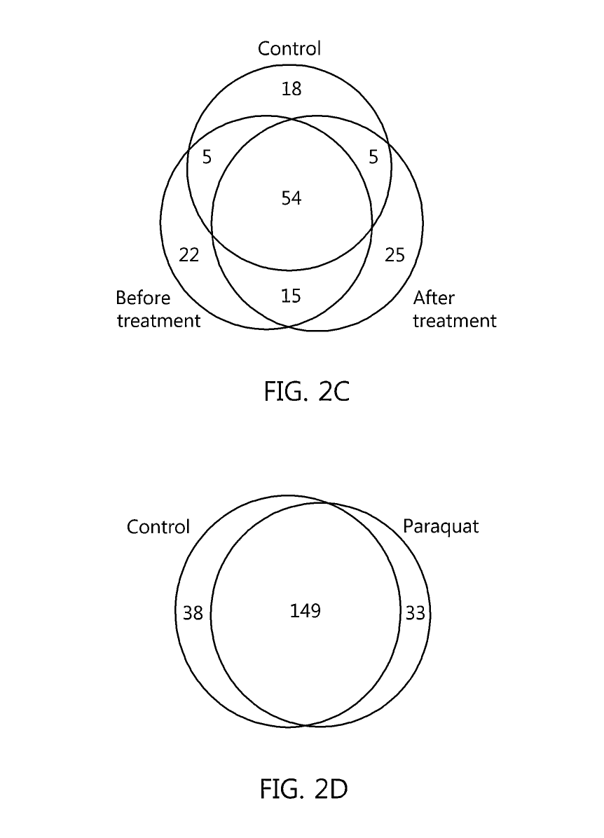

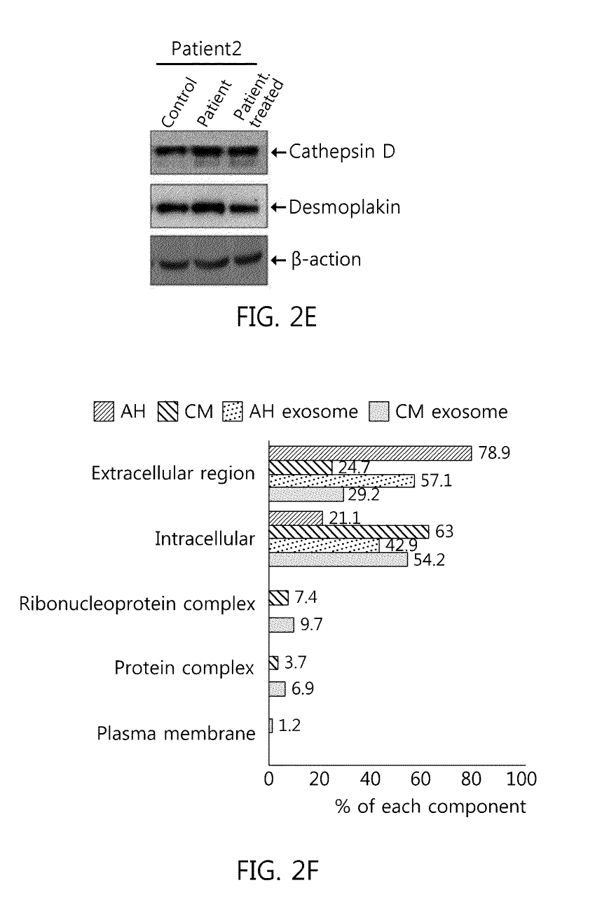

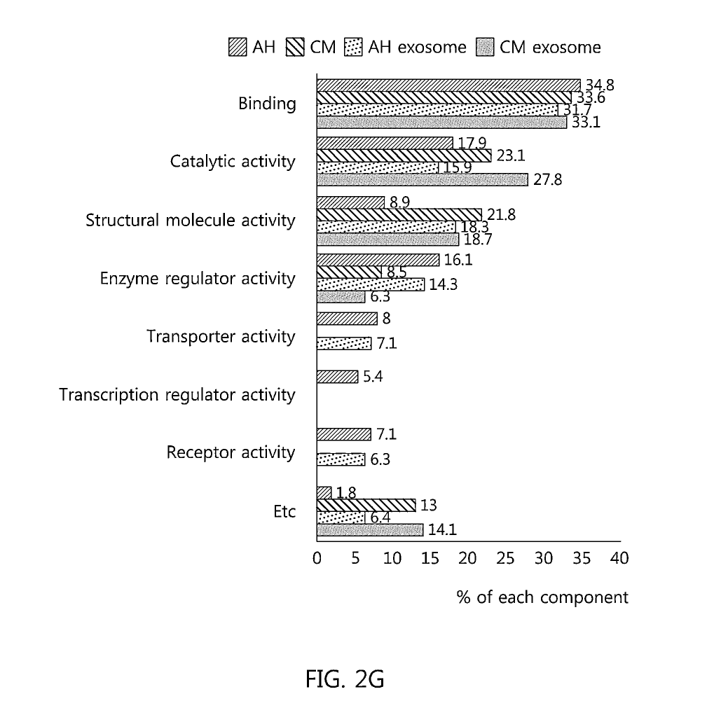

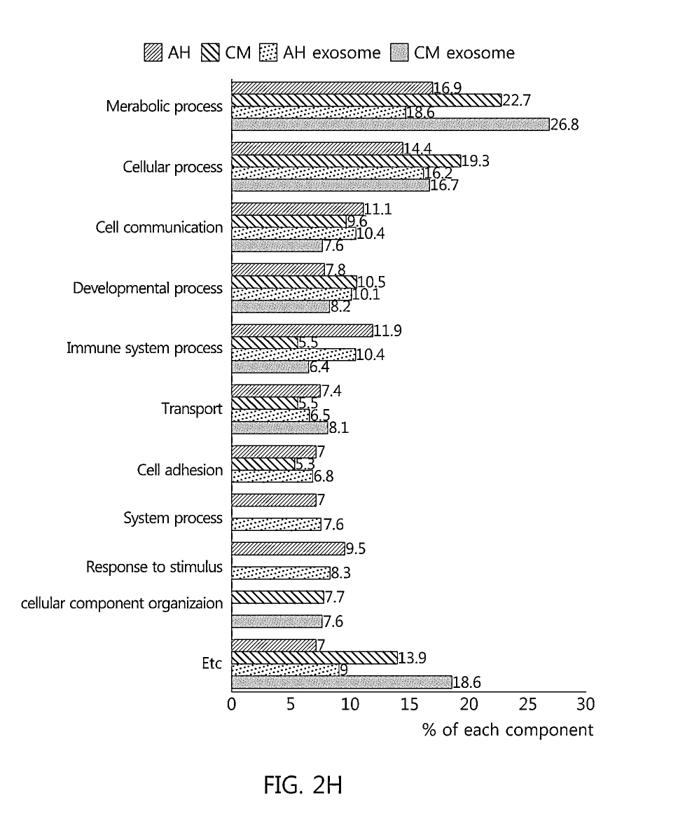

Four AH samples from two AMD patients before and after treatment (intravitreal injection of 0.5 mg ranibizumab) and their matched controls (sample set 1 in Table 1) were analyzed. We identified a total of 323 and 270 proteins in the AH of the two patients (FIG. 2A and Supplement Table 1), and 147 and 201 proteins in their respective matched controls. Despite a high degree of individual heterogeneity in AH protein expression (19), 180 proteins were common to both patient samples, and 173 proteins were common to both control samples. Many of the identified proteins have also been found in previous analyses of the AH (15, 19, 49). Albumin was the most abundant protein in the AH (50). The albumin concentration did not differ significantly between patients and controls, in contrast to the case in patients with uveitis, the pathology of which involves the breakdown of the blood-aqueous barrier (51).

To characterize the changes in the AH proteome associated with neovascular AMD and with its response to ranibizumab treatment, proteins that were upregulated by more than 1.5-fold in patients were analyzed and grouped by functional annotation in PANTHER (http://www.pantherdb.org/). Table 2 lists the identified proteins along with their biological process and putative functions. The expression of proteins involved in stress, oxidative stress, vision, aging, regulation of apoptosis, and cell differentiation was increased in patients and decreased after treatment. A total of 5 proteins were upregulated by more than 1.5-fold in both patients compared to controls (Table 2). The expression of heat shock protein β-1 (Hspβ-1) was upregulated by more than 1.5-fold in patient 1 compared to the controls. The expression of cathepsin D was upregulated in both patients (2.8 and 2.7-fold, respectively) and was decreased after treatment. The increased expression of cathepsin D and desmoplakin in patient 2 was confirmed by Western blot analysis (FIG. 2E).

TABLE 1
Summary of the demographic characteristics of age-related macular degeneration
(AMD) patients and control subjects.

| | Sample set 1: profiling | | | Sample set 2: profiling | | | Sample set 3: | | |
|---|---|---|---|---|---|---|---|---|---|
| | of total proteomes inAH^{a} | | | of exosomal proteins in AH | | | LC-MRM of AH | | |
| | AMD^{b} | | | AMD | | | AMD | | |
| Property | Before^{c} | After^{d} | Control | Before | After | Control | Before | After | Control |
| No. of AH samples | 2 | 2 | 2 | 9 | 9 | 9 | 15 | 15 | 15 |

| | | | | | | |
|---|---|---|---|---|---|---|
| Age (mean ± SD, years) | 70.5 ± 12.0 | 72.0 ± 7.1 | 69.8 ± 6.1 | 70.6 ± 4.0 | 70.3 ± 7.9 | 71.7 ± 6.4 |
| Sex (men:women) | 1:1 | 1:1 | 5:4 | 5:4 | 7:8 | 7:8 |
| Diabes mellitus (No.) | 0 | 0 | 2 | 2 | 3 | 3 |
| Hypension (No.) | 1 | 1 | 5 | 3 | 5 | 9 |

^{a}AH: aqueous humor;
^{b}AMD: age-related macular degeneration;
^{c}Before: before treatment with ranibizumab;
^{d}After: one month after treatment with ranibizumab

TABLE 2
List of proteins that were upregulated by more than 1.5-fold in the AH of
AMD patients compared to controls.

| Accession# | Protein Name | MW | Patient |
|---|---|---|---|
| 331999954 | keratin, type II cytoskeletal 4 | 56109.38 | Both |
| 131412228 | keratin, type I cytoskeletal 13 isoform b | 45808.64 | Both |
| 4503143 | cathepsin D preproprotein | 44523.66 | Both |
| 4506453 | retinol-binding protein 3 precursor | 135277.8 | Both |
| 58530840 | desmoplakin isoform I | 331567.8 | Both |
| 4502027 | serum albumin preproprotein | 69321.63 | 1 |
| 195972866 | keratin, type I cytoskeletal 10 | 58765.54 | 1 |
| 4557871 | serotransferrin precursor | 76999.66 | 1 |
| 4502101 | annexinA1 | 38690 | 1 |
| 5031839 | keratin, type II cytoskeletal 6A | 60008.32 | 1 |
| 131412225 | keratin, type I cytoskeletal 13 isoform a | 49527.42 | 1 |
| 55956899 | keratin, type I cytoskeletal 9 | 62026.66 | 1 |
| 12667788 | myosin-9 | 226390.6 | 1 |
| 119395750 | keratin, type II cytoskeletal 1 | 65998.94 | 1 |
| 189163542 | alpha-1-antitrypsin precursor | 46707.09 | 1 |
| 47132620 | keratin, type II cytoskeletal 2 epidermal | 65393.19 | 1 |
| 4504935 | keratin, type II cuticular Hb5 | 55766.54 | 1 |
| 24430192 | keratin, type I cytoskeletal 16 | 51236.25 | 1 |
| 169790841 | keratin, type II cuticular Hb3 | 54160.55 | 1 |
| 4507725 | transthyretin precursor | 15877.05 | 1 |
| 14917119 | keratin, type I cuticular Ha4 | 49392.29 | 1 |
| 16751921 | dermcidin preproprotein | 11276.83 | 1 |
| 7706635 | cornulin | 53501.61 | 1 |
| 410171504 | PREDICTED: homerin | 259153.5 | 1 |
| 4885049 | actin, alpha cardiac muscle 1 proprotein | 41991.89 | 1 |
| 4504517 | heat shock protein beta-1 | 22768.49 | 1 |
| 119395754 | keratin, type II cytoskeletal 5 | 62340.01 | 1 |
| 316659409 | actin, cytoplasmic 2 | 41765.8 | 1 |
| 62122917 | filaggrin-2 | 247926.3 | 1 |
| 427918083 | gelsolin isoform e | 81433.95 | 1 |
| 119703753 | keratin, type II cytoskeletal 6B | 60030.33 | 1 |
| 4506773 | protein S100-A9 | 13233.5 | 1 |
| 4503109 | cystatin-S precursor | 16203.97 | 1 |
| 4504183 | glutathione S-transferase P | 23341.02 | 1 |
| 167857790 | alpha-1-acid glycoprotein 1 precursor | 23524.81 | 1 |
| 91823274 | ectonucleotide pyrophosphatase/ | 105133 | 1 |
| | phosphodiesterase family member | | |
| | 2 isoform 1 preproprotein | | |
| 206597441 | aldehyde dehydrogenase, dimeric | 50362.9 | 2 |
| | NADP-preferring | | |
| 319996655 | c-myc promoter-binding protein-1 | 36904.86 | 2 |
| | isoform MBP-1 | | |
| 4557287 | angiotensinogen preproprotein | 53120.61 | 2 |
| 186910296 | haptoglobin isoform 2 preproprotein | 38427.33 | 2 |
| 67190748 | complement C4-A isoform 1 preproprotein | 192663.6 | 2 |
| 4557894 | lysozyme C precursor | 16526.29 | 2 |
| 262050546 | kininogen-1 isoform 3 precursor | 43793.63 | 2 |
| 57863246 | terminal uridylyltransferase 4 isoform b | 185047.3 | 2 |
| 21071030 | alpha-1B-glycoprotein precursor | 54219.66 | 2 |
| 109148552 | keratin, type II cytoskeletal 3 | 64377.6 | 2 |
| 115298678 | complement C3 precursor | 187029.3 | 2 |
| 388454162 | mitogen-activated protein kinase-binding | 134225.2 | 2 |
| | protein 1 isoform c | | |
| 37844908 | glyceraldehyde-3-phosphate dehydrogenase | 31527.96 | 2 |
| | isoform 2 | | |
| 28173560 | histone H4 | 11360.38 | 2 |
| 4557373 | biotinidase precursor | 61093.34 | 2 |
| 94538345 | keratin, type I cuticular Ha5 | 50328.61 | 2 |
| 50659080 | alpha-1-antichymotrypsin precursor | 47620.63 | 2 |
| 4504489 | histidine-rich glycoprotein precursor | 59540.94 | 2 |
| 89357932 | keratin, type II cytoskeletal 78 | 56830.53 | 2 |
| 4503009 | carboxypeptidase E preproprotein | 53117.17 | 2 |
| 5902010 | transcription termination factor, | 45749.07 | 2 |
| | mitochondrial precursor | | |
| 4504811 | junction plakoglobin | 81692.87 | 2 |
| 86788015 | EGF-containing fibulin-like extracellular | 54604.29 | 2 |
| | matrix protein 1 precursor | | |
| 4502067 | protein AMBP preproprotein | 38973.99 | 2 |
| 67782358 | complement factor B preproprotein | 85478.58 | 2 |
| 119392081 | complement factor I preproprotein | 65676.66 | 2 |
| 57242755 | calsyntenin-1 isoform 2 precursor | 108574.1 | 2 |
| 118442839 | complement factor H-related protein | 37625.96 | 2 |
| | 1 precursor | | |
| 167466233 | myosin light chain kinase family member 4 | 44479.64 | 2 |
| 16418467 | leucine-rich alpha-2-glycoprotein precursor | 38154.13 | 2 |
| 27477127 | keratin, type II cuticular Hb2 | 56616.38 | 2 |
| 60097902 | filaggrin | 434921.4 | 2 |
| 324021745 | vitamin D-binding protein isoform | 55041.09 | 2 |
| | 3 precursor | | |

1α: patient 1
2β: patient 2

Proteomic Analyses of the CM from Oxidatively Stressed ARPE-19 Cells

To determine which proteins of the AH from patients could be secreted from the RPE, we performed a comparative analysis of the proteins that were identified in profiling studies of six AH samples and the secretome analysis of the CM of ARPE-19 cells performed with LC-ESI-MS/MS.

Paraquat was added to ARPE-19 cells to mimic the heightened oxidative stress of the cellular environment in neovascular AMD. We identified a total of 334 proteins in the CM of ARPE-19 cells (FIG. 2B and Supplement Table 3). Table 3 shows the 25 proteins that were upregulated by more than 1.5-fold in the culture exposed to 400 μM paraquat for 24 h compared to the control culture along with their biological functions. Among the 334 secretome proteins that demonstrated altered expression in oxidatively stressed ARPE-19 cells, 65 proteins were found in the AH of one or both patients (Table 4). These proteins include actin, myosin 9, Hspβ-1, cytokeratin 9, and cathepsin D.

TABLE 3
List of the identities and biological functions of proteins that were upregulated
by more than 1.5-fold in the conditioned medium (CM) of ARPE-19
cells exposed to oxidative stress (400 μM paraquat for 24 h) compared to control CM.

| | Accession # | Protein Name | MW |
|---|---|---|---|
| Coagulation and | | | |
| complement- | | | |
| involved protein | | | |
| 1 | GI 189163542 | alpha-1-antitrypsin precursor [Homo sapiens] | 46707.09 |
| 2 | 4502261 | antithrombin-III precursor [Homo sapiens] | 52568.98 |
| 3 | 259155306 | plasminogen activator inhibitor 1 isoform 2 precursor [Homo sapiens] | 43376.22 |
| 4 | 40317626 | thrombospondin-1 precursor [Homo sapiens] | 129299.2 |
| Wound healing | | | |
| 1 | 58530842 | desmoplakin isoform II [Homo sapiens] | 259954.2 |
| 2 | 189083782 | gelsolin isoform c [Homo sapiens] | 81890.13 |
| 3 | 189163542 | alpha-1-antitrypsin precursor [Homo sapiens] | 46707.09 |
| 4 | 4502261 | antithrombin-III precursor [Homo sapiens] | 52568.98 |
| 5 | 259155306 | plasminogen activator inhibitor 1 isoform 2 precursor [Homo sapiens] | 43376.22 |
| 6 | 63252906 | tropomyosin alpha-1 chain isoform 7 [Homo sapiens] | 32796.73 |
| regeneration | | | |
| 1 | 4501989 | alpha-fetoprotein precursor [Homo sapiens] | 68633.09 |
| 2 | 189083782 | gelsolin isoform c [Homo sapiens] | 81890.13 |
| 3 | 259155306 | plasminogen activator inhibitor 1 isoform 2 precursor [Homo sapiens] | 43376.22 |
| Aging | | | |
| 1 | 189083782 | gelsolin isoform c [Homo sapiens] | 81890.13 |
| 2 | 205277441 | thyroxine-binding globulin precursor [Homo sapiens] | 46294.67 |
| 3 | 259155306 | plasminogen activator inhibitor 1 isoform 2 precursor [Homo sapiens] | 43376.22 |
| Cell adhesion | | | |
| 1 | 189083782 | gelsolin isoform c [Homo sapiens] | 81890.13 |
| 2 | 40317626 | thrombospondin-1 precursor [Homo sapiens] | 129299.2 |
| 3 | 63252906 | tropomyosin alpha-1 chain isoform 7 [Homo sapiens] | 32796.73 |
| Regulation of | | | |
| body fluid levels | | | |
| 1 | 189163542 | alpha-1-antitrypsin precursor [Homo sapiens] | 46707.09 |
| 2 | 4502261 | antithrombin-III precursor [Homo sapiens] | 52568.98 |
| 3 | 259155306 | plasminogen activator inhibitor 1 isoform 2 precursor [Homo sapiens] | 43376.22 |
| Protein folding | | | |
| 1 | 5453603 | T-complex protein 1 subunit beta isoform 1 [Homo sapiens] | 57452.26 |
| 2 | 13325075 | sulfhydryl oxidase 1 isoform a [Homo sapiens] | 82525.73 |
| 3 | 56550043 | sarcolemmal membrane-associated protein [Homo sapiens] | 93145.48 |

TABLE 4
Possible secreted proteins identified in the AH of patients compared to CM
from oxidatively stressed ARPE-19 cells.

| No. | Accession # | EntrezID | Protein Name | M.W. |
|---|---|---|---|---|
| 1 | 115298678 | 718 | complement C3 precursor [Homo sapiens] | 187029.3 |
| 2 | 4501989 | 174 | alpha-fetoprotein precursor [Homo sapiens] | 68633.09 |
| 3 | 324021745 | #N/A | vitamin D-binding protein isoform 3 precursor [Homo sapiens] | 55041.09 |
| 4 | 119703753 | 3854 | keratin, type II cytoskeletal 6B [Homo sapiens] | 60030.33 |
| 5 | 162809334 | 5858 | pregnancy zone protein precursor [Homo sapiens] | 163759.1 |
| 6 | 4502027 | 213 | setum albumin preproprotein [Homo sapiens] | 69321.63 |
| 7 | 122937400 | 55714 | teneurin-3 [Homo sapiens] | 300757.7 |
| 8 | 4502261 | 462 | antithrombin-III precursor [Homo sapiens] | 52568.98 |
| 9 | 189163542 | 5265 | alpha-1-antitrypsin precursor [Homo sapiens] | 46707.09 |
| 10 | 316659409 | #N/A | actin, cytoplasmic 2 [Homo sapiens] | 41765.8 |
| 11 | 124256476 | 1608 | diacylglycerol kinase gamma isoform 1 [Homo sapiens] | 89065.98 |
| 12 | 24430192 | 3868 | keratin, type I cytoskeletal 16 [Homo sapiens] | 51236.25 |
| 13 | 4507509 | 7076 | metalloproteinase inhibitor 1 precursor [Homo sapiens] | 23155.55 |
| 14 | 70778918 | 3698 | inter-alpha-trypsin inhibitor heavy chain H2 [Homo sapiens] | 106396.8 |
| 15 | 109148552 | 3850 | keratin, type II cytoskeletal 3 [Homo sapiens] | 64377.6 |
| 16 | 4502101 | 301 | annexin Al [Homo sapiens] | 38690 |
| 17 | 156119625 | 3697 | inter-alpha-trypsin inhibitor heavy chain H1 isoform a [Homo sapiens] | 101325.8 |
| 18 | 291575128 | 3945 | L-lactate dehydrogenase B chain [Homo sapiens] | 36615.16 |
| 19 | 324021738 | #N/A | amyloid beta A4 protein isoform h precursor [Homo sapiens] | 84986.14 |
| 20 | 15431310 | 3861 | keratin, type I cytoskeletal 14 [Homo sapiens] | 51589.5 |
| 21 | 4507467 | 7045 | transforming growth factor-beta-induced protein ig-h3 precursor [Homo sapiens] | 74634.1 |
| 22 | 4503471 | 1915 | elongation factor 1-alpha 1 [Homo sapiens] | 50109.18 |
| 23 | 5729877 | 3312 | heat shock cognate 71 kDa protein isoform 1 [Homo sapiens] | 70854.35 |
| 24 | 331999954 | #N/A | keratin, type II cytoskeletal 4 [Homo sapiens] | 56109.38 |
| 25 | 156523970 | 197 | alpha-2-HS-glycoprotein [Homo sapiens] | 39315.71 |
| 26 | 119395750 | 3848 | keratin, type II cytoskeletal 1 [Homo sapiens] | 65998.94 |
| 27 | 47132620 | 3849 | keratin, type II cytoskeletal 2 epidermal [Homo sapiens] | 65393.19 |
| 28 | 66932947 | 2 | alpha-2-macroglobulin precursor [Homo sapiens] | 163188.3 |
| 29 | 156627579 | 7123 | tetranectin precursor [Homo sapiens] | 22522.28 |
| 30 | 39725934 | 5176 | pigment epithelium-derived factor precursor [Homo sapiens] | 46283.37 |
| 31 | 11321593 | 3489 | insulin-like growth factor-binding protein 6 precursor [Homo sapiens] | 25306.17 |
| 32 | 4503107 | 1471 | cystatin-C precursor [Homo sapiens] | 15789.08 |
| 33 | 126012571 | 3339 | basement membrane-specific heparan sune proteoglycan core protein | 468533.1 |
| | | | precursor [Homo sapiens] | |
| 34 | 5031839 | 3853 | keratin, type II cytoskeletal 6A [Homo sapiens] | 60008.32 |
| 35 | 27477127 | 3888 | keratin, type II cuticular Hb2 [Homo sapiens] | 56616.38 |
| 36 | 116488398 | 3882 | keratin, type I cuticular Ha2 [Homo sapiens] | 50310.25 |
| 37 | 4557871 | 7018 | serotransferrin precursor [Homo sapiens] | 76999.66 |
| 38 | 55956899 | 3857 | keratin, type I cytoskeletal 9 [Homo sapiens] | 62026.66 |
| 39 | 4504919 | 3856 | keratin, type II cytoskeletal 8 [Homo sapiens] | 53671.19 |
| 40 | 16507237 | 3309 | 78 kDa glucose-regulated protein precursor [Homo sapiens] | 72288.52 |
| 41 | 14917115 | 3881 | keratin, type I cuticular Ha1 [Homo sapiens] | 47207.11 |
| 42 | 5031863 | 3959 | galectin-3-binding protein [Homo sapiens] | 65289.4 |
| 43 | 57242755 | 22883 | calsyntenin-1 isoform 2 [Homo sapiens] | 108574.1 |
| 44 | 110349788 | 55870 | probable histone-lysine N-methyltransferase ASH1L [Homo sapiens] | 332W0.7 |
| 45 | 4504935 | 3891 | keratin, type II cuticular Hb5 [Homo sapiens] | 55766.54 |
| 46 | 195972866 | 3858 | keratin, type I cytoskeletal 10 [Homo sapiens] | 58765.54 |
| 47 | 12667788 | 4627 | myosin-9 [Homo sapiens] | 226390.6 |
| 48 | 119395754 | 3852 | keratin, type II cytoskeletal 5 [Homo sapiens] | 62340.01 |
| 49 | 31657092 | 154664 | ATP-binding cassette sub-family A member 13 [Homo sapiens] | 575792.9 |
| 50 | 16751921 | 117159 | dermcidin preproprotein [Homo sapiens] | 11276.83 |
| 51 | 4557701 | 3872 | keratin, type I cytoskeletal 17 [Homo sapiens] | 48076.11 |
| 52 | 4507511 | 7077 | metalloproteinase inhibitor 2 precursor [Homo sapiens] | 24383.13 |
| 53 | 55925576 | 3485 | insulin-like growth factor-binding protein 2 precursor [Homo sapiens] | 35114.41 |
| 54 | 169790853 | 3887 | keratin, type II cuticular Hb1 [Homo sapiens] | 54892.8 |
| 55 | 5902010 | 7978 | transcription termination factor, mitochondrial precursor [Homo sapiens] | 45749.07 |
| 56 | 94538345 | 3886 | keratin, type I cuticular Ha5 [Homo sapiens] | 50328.61 |
| 57 | 4503635 | 2147 | prothrombin preproprotein [Homo sapiens] | 69992.2 |
| 58 | 61743954 | 79026 | neuroblast differentiation-associated protein AHNAK isoform 1 [Homo sapiens] | 628705.2 |
| 59 | 4503143 | 1509 | cathepsin D preproprotein [Homo sapiens] | 44523.66 |
| 60 | 24430190 | 3866 | keratin, type I cytoskeletal 15 [Homo sapiens] | 49167.11 |
| 61 | 242332527 | 65250 | hypothetical protein LOC65250 [Homo sapiens] | #N/A |
| 62 | 320461711 | #N/A | peroxiredoxin-1 [Homo sapiens] | 22096.28 |
| 63 | 4557894 | 4069 | lysozyme C precursor [Homo sapiens] | 16526.29 |
| 64 | 50345984 | 498 | ATP synthase subunit alpha, mitochondrial precursor [Homo sapiens] | 59713.73 |
| 65 | 94536793 | 79739 | tubulin polyglutamylase TTLL7 [Homo sapiens] | 102933.8 |

The Isolation and Characterization of Exosomes from the AH of Patients and CM from ARPE-19 Cell Culture

For further verification of the proteins secreted by the RPE of patients and to narrow down the list of proteins that could be most relevant to our study, we analyzed the secretory vesicles in AH and CM. Among the microvesicles that cells secrete to the extracellular spaces, we chose to focus on exosomes because many previous studies have reported their biological importance in body fluids (30, 31, 36, 38). Exosomes have not been found in patients' AH to date. Treatment with 400 μM paraquat for 24 h did not induce cell death or apoptosis in ARPE-19 cells, as determined by FACS analysis, whereas concentrations higher than 500 μM were cytotoxic (data not shown). This result confirms that the harvested exosomes and exosomal release of proteins were not produced as a consequence of cell death.

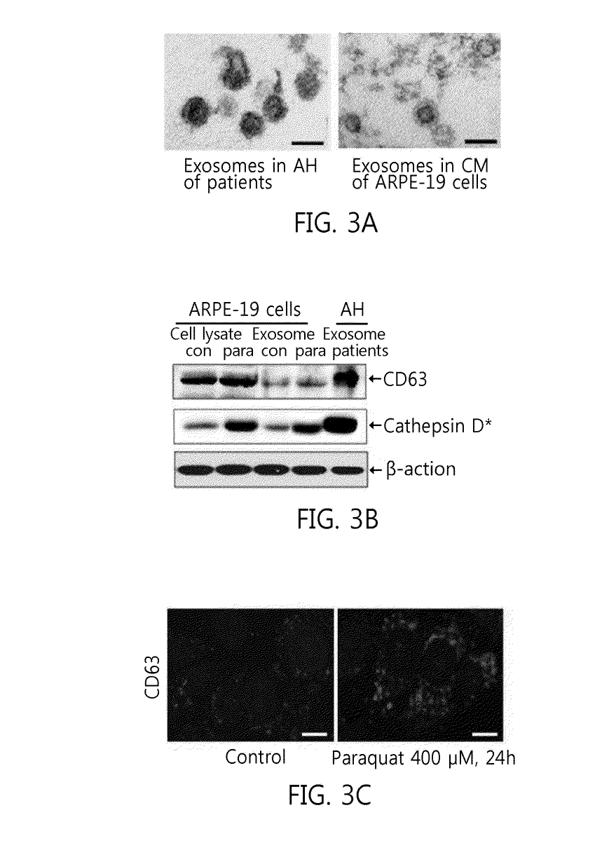

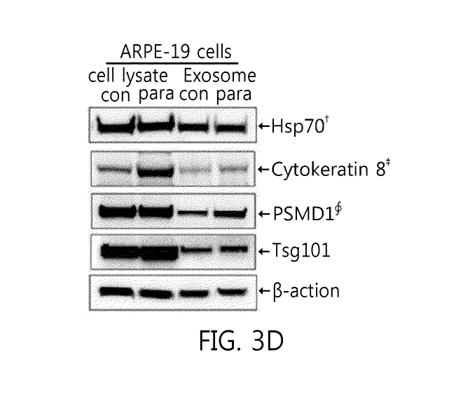

The exosome pellets from the AH of patients and CM from ARPE-19 cell culture were obtained with ExoQuick™ Exosome Precipitation Solution according to the manufacturer's protocol, as described in the Experimental Procedures.TEM revealed that exosomes in the AH of patients and CM from paraquat-treated ARPE-19 cells appeared as homogeneous round-shaped membrane vesicles with diameters of 50-100 nm (FIG. 3A). To further characterize the exosomes, we used Western blot analysis to examine whether common exosomal marker proteins were present in the purified exosome pellet. The most widely used markers include tetraspannins (CD9, CD63, CD81, CD82) and Hsp70, and Western blot analysis is widely used for rapid confirmation of exosome presence (38). Equivalent amounts of proteins from the exosomes of AH of patients, exosomes from the CM of ARPE-19 cells, and the total cell lysates of ARPE-19 cells were loaded on the same gel. We detected CD63 in the exosomes from the AH of patients and CM of ARPE-19 cells (FIG. 3B). The AH contained a large quantity of exosomes, which was reflected by the marked CD63 content found in this fraction by Western blot analysis. We also noted that the level of cathepsin D was high in exosomes from the AH of patients compared to the exosomes from CM or cell lysates. Increased expression of globular CD63 was also detected in oxidatively stressed ARPE-19 cells compared with control cells (FIG. 3C). We noted that the overall amount of exosomes increased in the CM of paraquat-treated ARPE-19 cells compared with the control cells (data not shown). We also examined whether any of the proteins that were found in the AH of patients (supplement Table 1) were present in the exosomes from these samples. We confirmed the expression of Hsp70, cytokeratin 8, and 26S proteasome non-ATPase subunit 1 (PSMD1), along with tumor susceptibility gene 101 (Tsg101), which is a component of ESCRT (endosomal sorting complexes required for transport) in exosomes from CM (FIG. 3D). Thus, we hypothesized that the proteins in the AH of patients might be secreted from the RPE via exosomes. We investigated this hypothesis further by performing proteomic profiling of all exosomal proteins of AH and CM.

Proteomic Profiling and Comparative Proteomic Analysis of Exosomes Derived from the AH of Patients and CM from ARPE-19 Cell Cultures

To further explore the possibility that some of the proteins in the AH were secreted via exosomes, the exosome proteome obtained from AH patients and CM from ARPE-19 cells were analyzed using a label-free semi-quantitative mass spectrometry system (LC-ESI-MS/MS). In total, we identified 145 and 220 proteins that were detected more than 2 times in triplicate experiments in exosomes from the AH of patients and CM of ARPE-19 cell culture, respectively (FIGS. 2C and 2D and supplement Table 4 and 5). These proteins included members of the armexin family (annexin A1, A2, A3, A4, A5), the heat shock protein family (Hsp70, 90 alpha and beta), cytoskeletal proteins (cytokeratin 1, 5, 7, 8, 9, 18, and 19), chaperone proteins, members of the proteasome-ubiquitin pathway, proteases and protease inhibitors, coagulation and complement cascades, proteins involved in transport and metabolism, signaling molecules, and housekeeping proteins (e.g., GAPDH, glyceraldehyde 3-phosphate dehydrogenase). The 25 proteins that are most often identified in exosomes (ExoCarta, exocarta.org) were also found in the exosomes in this study. We also identified several new proteins that had not been previously described in exosomes by examining exosomes from the AH of patients (Supplement Table 6). In addition to cytokeratin 8, cathepsin D, 26S proteasome non-ATPase subunit 1, and Hsp 70, which were found in the exosomes from the AH and/or CM by Western blot analysis (FIGS. 3B and 3D), actin (smooth muscle), myosin 9, Hspβ-1, cytokeratin 9, vitronectin, and retinol-binding protein 4 were also found in the exosomes of AH or CM by proteomic analysis. Table 5 shows the exosomal proteins that were upregulated by more than 1.5-fold in the AH of patients or CM from ARPE-19 cells compared to controls. Among the 363 and 145 proteins identified in the AH of patients and exosomes from the AH of patients, respectively, 78 proteins appeared in both samples (Table 6). The molecular function, biological process, cellular component, and pathway annotations of these proteins were classified using PANTHER software. As shown in FIGS. 2F, 2G, and 2H, the majority of these proteins were involved in metabolic processes, immune system processes, response to stimulus, or developmental processes. The AH proteins were associated with 9 types of activities, mainly binding activity, catalytic activity, enzyme regulator activity, and structural molecular activity.

TABLE 5
Exosomal proteins identified by LC-ESI-MS/MS analysis increased by more
than 1.5-fold in the AH of patients compared to the AH of control subjects (Upper part). Exosomal
proteins identified by LC-ESI-MS/MS analysis increased by more than 1.5-fold in the CM from
ARPE-19 cell cultures exposed to oxidative stress compared to CM from control cultures (Bottom part).

| No. | Accession # | EntrezID | Protein Name | MW |
|---|---|---|---|---|
| 1 | 27436940 | 5649 | reelin isoform b [Homo sapiens] | 387950.7 |
| 2 | 4504489 | 3273 | histidine-rich glycoprotein precursor [Homo sapiens] | 59540.94 |
| 3 | 56550061 | 11314 | CMRF35-like molecule 8 [Homo sapiens] | 33179.89 |
| 4 | 50659080 | 12 | alpha-1-antichymotrypsin precursor [Homo sapiens] | 47620.63 |
| 5 | 144226251 | 1116 | chitinase-3-like protein 1 precursor [Homo sapiens] | 42598.43 |
| 6 | 11761629 | 2243 | fibrinogen alpha chain isoform alpha preproprotein [Homo sapiens] | 69713.77 |
| 7 | 300388183 | #N/A | 26S proteasome non-ATPase regulatory subunit 1 isoform 2 [Homo sapiens] | 1021929 |
| 8 | 296080754 | 2244 | fibrinogen beta chain isoform 2 preproprotein [Homo sapiens] | 49923.16 |
| 9 | 47132557 | 2335 | fibronectin isoform 1 preproprotein [Homo sapiens] | 272157.3 |
| 10 | 4507725 | 7276 | transthyretin precursor [Homo sapiens] | 15877.05 |
| 11 | 41393602 | 716 | complement C1s subcomponent precursor [Homo sapiens] | 76634.85 |
| 12 | 145309328 | 89932 | papilin [Homo sapiens] | 134753.8 |
| 13 | 11321593 | 3489 | insulin-like growth factor-binding protein 6 precursor [Homo sapiens] | 25306.17 |
| 14 | 14043022 | 4141 | methionyl-tRNA synthetase, cytoplasmic [Homo sapiens] | 101052 |
| 15 | 34734062 | 2192 | fibula-1 isoform C precursor [Homo sapiens] | 7441202 |
| 16 | 31657092 | 154664 | ATP-binding cassette sub-family A member 13 [Homo sapiens] | 575792.9 |
| 17 | 115647957 | 64222 | totsin-3A precursor [Homo sapiens] | 46169.39 |
| 18 | 70906439 | 2266 | fibrinogen gamma chain isoform gamma-B precursor [Homo sapiens] | 51478.88 |
| 19 | 324021745 | #N/A | vitamin D-binding protein isoform 3 precursor [Homo sapiens] | 55041.09 |
| 20 | 66347875 | 715 | complement C1r subcomponent precursor [Homo sapiens] | 80147.95 |
| 21 | 126012571 | 3339 | basement membrane-specific heparan sulfate proteoglycan core protein | 468533.1 |
| | | | precursor [Homo sapiens] | |
| 22 | 4505881 | 5340 | plasminogen isoform 1 precursor [Homo sapiens] | 90510.23 |
| 23 | 4502027 | 213 | setum albumin preproprotein [Homo sapiens] | 69321.63 |
| 24 | 4505529 | 5005 | alpha-1-acid glycoprotein 2 precursor [Homo sapiens] | 23587.64 |
| 25 | 4557321 | 335 | apolipoprotein A-I preproprotein [Homo sapiens] | 30758.94 |
| 26 | 55743122 | 5950 | retinol-binding protein 4 precursor [Homo sapiens] | 22995.26 |
| 27 | 4557287 | 183 | angiotensinogen preproprotein [Homo sapiens] | 53120.61 |
| 28 | 61743954 | 79026 | neuroblast differentiation-associated protein AHNAK isoform 1 [Homo sapiens] | 628705.2 |
| 29 | 186)10296 | 3240 | haptoglobin isoform 2 preproprotein [Homo sapiens] | 38427.33 |
| 30 | 189181750 | 5926 | transcription factor HIVEP3 isoform b [Homo sapiens] | 259173.4 |
| 31 | 187608545 | 55165 | centrosomal protein of 55 kDa [Homo sapiens] | 54115.3 |
| 32 | 71274148 | 57480 | pleckstrin homology domain-ccntaining family G member 1 [Homo sapiens] | 155343 |
| 33 | 42734397 | 90025 | ubiquitin-conjugating enzyme E2C-binding protein [Homo sapiens] | 43628.38 |
| 34 | 46275837 | 23019 | CCR4-NOT transcription complex subunit 1 isoform b [Homo sapiens] | 173679.7 |
| 35 | 166235903 | 714 | complement C1q subcomponent subunit C precursor [Homo sapiens] | 25757.13 |
| 36 | 57242755 | 22883 | calsyntenin-1 isoform 2 [Homo sapiens] | 108574.1 |
| 37 | 122891870 | 375056 | melanoma inhibitory activity protein 3 precursor [Homo sapiens] | 213568.8 |
| 38 | 19743801 | 10782 | neurotrophin receptor-interacting factor homolog isoform c [Homo sapiens] | 74131.09 |
| 39 | 122937261 | 114800 | coiled-coil domain-containing protein 85A [Homo sapiens] | 59938.69 |
| 40 | 15431310 | 3861 | keratin, type I cytoskeletal 14 [Homo sapiens] | 51589.5 |
| 41 | 67190748 | 720 | complement C4-A preproprotein [Homo sapiens] | 192663.6 |
| 42 | 40806187 | 170692 | A disintegrin and metalloproteinase with thrombospondin motifs 18 | 135078.6 |
| | | | preproprotein [Homo sapiens] | |
| 43 | 16933540 | 2191 | seprase [Homo sapiens] | 87656.75 |
| 44 | 7662424 | 22869 | zinc finger protein 510 [Homo sapiens] | 79091.67 |
| 45 | 225735595 | 79657 | RNA polymerase II-associated protein 3 isoform 3 [Homo sapiens] | 57072.68 |

TABLE 6
List of common exosomal proteins identified in both the AH and exosomes isolated from the AH.

| No. | Accession # | EntrezID | Protein Name | MW |
|---|---|---|---|---|
| 1 | 4502027 | 213 | serum albumin preproprotein [Homo sapiens] | 69321.63 |
| 2 | 4557871 | 7018 | serotransferrin precursor [Homo sapiens] | 76999.66 |
| 3 | 119395750 | 3848 | keratin, type II cytoskeletal 1 [Homo sapiens] | 65998.94 |
| 4 | 55956899 | 3857 | keratin, type I cytoskeletal 9 [Homo sapiens] | 62026.66 |
| 5 | 119395754 | 3852 | keratin, type II cytoskeletal 5 [Homo sapiens] | 62340.01 |
| 6 | 15431310 | 3861 | keratin, type I cytoskeletal 14 [Homo sapiens] | 51589.5 |
| 7 | 47132620 | 3849 | keratin, type II cytoskeletal 2 epidermal [Homo sapiens] | 65393.19 |
| 8 | 195972866 | 3858 | keratin, type I cytoskeletal 10 [Homo sapiens] | 58765.54 |
| 9 | 189163542 | 5265 | alpha-1-antitrypsin precursor [Homo sapiens] | 46707.09 |
| 10 | 115298678 | 718 | complement C3 precursor [Homo sapiens] | 187029.3 |
| 11 | 4557485 | 1356 | ceruloplasmin precursor [Homo sapiens] | 122127.6 |
| 12 | 4506453 | 5949 | retinol-binding protein 3 precursor [Homo sapiens] | 135277.8 |
| 13 | 66932947 | 2 | alpha-2-macroglobulin precursor [Homo sapiens] | 163188.3 |
| 14 | 67190748 | 720 | complement C4-A preprcprotein [Homo sapiens] | 192663.6 |
| 15 | 24430192 | 3868 | keratin, type I cytoskeletal 16 [Homo sapiens] | 51236.25 |
| 16 | 39725934 | 5176 | pigment epithelium-derived factor precursor [Homo sapiens] | 46283.37 |
| 17 | 189083782 | 2934 | gelsolin isoform c [Homo sapiens] | 81890.13 |
| 18 | 11321561 | 3263 | hemopexin precursor [Homo sapiens] | 51643.32 |
| 19 | 4557325 | 348 | apolipoprotein E precursor [Homo sapiens] | 36131.79 |
| 20 | 4557701 | 3872 | keratin, type I cytoskeletal 17 [Homo sapiens] | 48076.11 |
| 21 | 4557321 | 335 | apolipoprotein A-I preproprotein [Homo sapiens] | 30758.94 |
| 22 | 67782358 | 629 | complement factor B preproprotein [Homo sapiens] | 85478.58 |
| 23 | 71773110 | 337 | apolipoprotein A-IV precursor [Homo sapiens] | 45344.51 |
| 24 | 167857790 | 5004 | alpha-1-acid glycoprotein 1 precursor [Homo sapiens] | 23524.81 |
| 25 | 57864582 | 388697 | homerin [Homo sapiens] | 282225.7 |
| 26 | 4507725 | 7276 | transthyretin precursor [Homo sapiens] | 15877.05 |
| 27 | 4503143 | 1509 | cathepsin D preproprotein [Homo sapiens] | 44523.66 |
| 28 | 4502261 | 462 | antithrombin-III precursor [Homo sapiens] | 52568.98 |
| 29 | 4505881 | 5340 | plasminogen isoform 1 precursor [Homo sapiens] | 90510.23 |
| 30 | 86788132 | 2202 | EGF-containing fibulin-like extracellular matrix protein 1 | 54604.29 |
| | | | precursor [Homo sapiens] | |
| 31 | 6006001 | 2878 | glutathione peroxidase 3 precursor [Homo sapiens] | 25385.96 |
| 32 | 50659080 | 12 | alpha-1-antichymotrypsin precursor [Homo sapiens] | 47620.63 |
| 33 | 66346689 | 27122 | dickkopf-related protein 3 precursor [Homo sapiens] | 38365.15 |
| 34 | 73858570 | 710 | plasma protease C1 inhibitor precursor [Homo sapiens] | 55119.49 |
| 35 | 4826762 | 3240 | haptoglobin isoform 1 preproprotein [Homo sapiens] | 45176.59 |
| 36 | 262050546 | 3827 | kininogen-1 isoform 3 [Homo sapiens] | 43793.63 |
| 37 | 4557287 | 183 | angiotensinogen preproprotein [Homo sapiens] | 53120.61 |
| 38 | 153266841 | 350 | beta-2-glycoprotein 1 precursor [Homo sapiens] | 38272.67 |
| 39 | 156523970 | 197 | alpha-2-HS-glycoprotein [Homo sapiens] | 39315.71 |
| 40 | 4505529 | 5005 | alpha-1-acid glycoprotein 2 precursor [Homo sapiens] | 23587.64 |
| 41 | 61743954 | 79026 | neuroblast differentiation-associated protein AHNAK isoform 1 | 628705.2 |
| | | | [Homo sapiens] | |
| 42 | 7657419 | 26254 | opticin precursor [Homo sapiens] | 37237.39 |
| 43 | 32171249 | 5730 | prostaglandin-H2D-isomerase [Homo sapiens] | 21015.35 |
| 44 | 34734068 | 2192 | fibulin-1 isoform A precursor [Homo sapiens] | 61538.3 |
| 45 | 4503635 | 2147 | prothrombin preproprotein [Homo sapiens] | 69992.2 |
| 46 | 88853069 | 7448 | vitronectin precursor [Homo sapiens] | 54271.23 |
| 47 | 55743122 | 5950 | retinol-binding protein 4 precursor [Homo sapiens] | 22995.26 |
| 48 | 118442839 | 3078 | complement factor H-related protein 1 precursor [Homo sapiens] | 37625.96 |
| 49 | 4504489 | 3273 | histidine-rich glycoprotein precursor [Homo sapiens] | 59540.94 |
| 50 | 144226251 | 1116 | chitinase-3-like protein 1 precursor [Homo sapiens] | 42598.43 |
| 51 | 11321593 | 3489 | insulin-like growth factor-binding protein 6 precursor [Homo sapiens] | 25306.17 |
| 52 | 5802984 | 11041 | N-acetyllactosaminide beta-1,3-N-acetylglucosaminyltransferase [Homo sapiens] | 47088.94 |
| 53 | 262050538 | 3700 | inter-alpha-trypsin inhibitor heavy chain H4 isoform 2 precursor [Homo sapiens] | 99795.37 |
| 54 | 4502147 | 334 | amyloid-like protein 2 isoform 1 [Homo sapiens] | 86900.28 |
| 55 | 5031863 | 3959 | galectin-3-binding protein [Homo sapiens] | 65289.4 |
| 56 | 111125011 | 11197 | wnt inhibitory factor 1 precursor [Homo sapiens] | 41499.8 |
| 57 | 126012571 | 3339 | basement membrane-specific heparan sulfate proteoglycan core protein | 468533.1 |
| | | | precursor [Homo sapiens] | |
| 58 | 4557733 | 4053 | latent-transforming growth factor beta-binding protein 2 precursor | 194922.3 |
| | | | [Homo sapiens] | |
| 59 | 214010185 | 334 | amyloid-like protein 2 isoform 4 [Homo sapiens] | 59114.2 |
| 60 | 62739186 | 3075 | complement factor H isoform a precursor [Homo sapiens] | 138978.4 |
| 61 | 31657092 | 154664 | ATP-binding cassette sub-family A member 13 [Homo sapiens] | 575792.9 |
| 62 | 11386161 | 1298 | collagen alpha-2(IX) chain precursor [Homo sapiens] | 65090.96 |
| 63 | 14589893 | 1002 | cadherin4 preproprotein [Homo sapiens] | 100217.9 |
| 64 | 256818774 | 55779 | WD repeat-containing protein 52 isoform 1 [Homo sapiens] | 213729.9 |
| 65 | 7662424 | 22869 | zinc finger protein 510 [Homo sapiens] | 79091.67 |
| 66 | 154146262 | 8857 | IgGFc-bincling protein precursor [Homo sapiens] | 571640.9 |
| 67 | 27436940 | 5649 | reelin isoform b [Homo sapiens] | 387950.7 |
| 68 | 281485550 | 2200 | fibrillin-1 precursor [Homo sapiens] | 312082 |
| 69 | 4557779 | 4653 | myocilin precursor [Homo sapiens] | 56936.61 |
| 70 | 260064050 | 5345 | alpha-2-antiplasmin isoform b precursor [Homo sapiens] | 47876.72 |
| 71 | 190341024 | 8404 | SPARC-like protein 1 precursor [Homo sapiens] | 75161.32 |
| 72 | 4507467 | 7045 | transforming growth factor-beta-induced protein ig-h3 precursor [Homo sapiens] | 74634.1 |
| 73 | 4504165 | 2934 | gelsolin isoform a precursor [Homo sapiens] | 85644.25 |
| 74 | 34734066 | 2192 | fibula-1 isoform D precursor [Homo sapiens] | 77190A6 |
| 75 | 42734397 | 90025 | ubiquitin conjugating enzyme E2C-binding protein [Homo sapiens] | 43628.38 |
| 76 | 209862791 | 7643 | zinc finger protein 90 [Homo sapiens] | 6901277 |
| 77 | 74271828 | 80298 | mTERF domain-containing protein 3, mitochondrial precursor [Homo sapiens] | 44385.62 |
| 78 | 112799847 | 6262 | ryanodine receptor 2 [Homo sapiens] | 564205.3 |

Generation of the Target Proteins for LC-MRM

In the whole-proteome profiling of the AH of two patients, candidate proteins were identified by comparison with data from the AH of controls (Table 2 and Supplement Table 1). A total of 413 identified proteins were searched against previously published studies of proteins or genes identified in AH, RPE cell culture media, or proteins found in donor eyes with AMD (11, 52-54) to determine their relevance to AMD or AMD-related conditions such as oxidative stress. Target proteins for LC-MRM were selected based on two criteria: 1) the protein must be present in the AH profile and in one of the following three profiles: CM from ARPE-19 cell culture, exosomes from AH, or exosomes from CM (Table 7); and 2) the peptides of a target protein must be frequently observed in MS scans because these proteins are easily observed in LC-MRM assays. Based on these criteria, 12 candidate proteins considered to be originated from the RPE of patients and present in the AH of patients with neovascular AMD were selected and used in a quantitative LC-MRM assay to measure the levels of these proteins in the AH samples from AMD patients. The selected proteins were desmoplakin; actin (aortic smooth muscle); myosin 9; Hsp70; Hspβ-1; cytokeratins 8, 9, and 14; vitronectin; 26S proteasome non-ATPase subunit 1 (PSMD1); cathepsin D; and retinol-binding protein 4.

TABLE 7
The selection of 12 target proteins which are common to the investigated
sample sets for LC-MRM.

| | AH | Exosomes | CM from | Exosomes from |
|---|---|---|---|---|
| | profiling | from AH | ARPE-19 cells | CM of ARP-19 cells |
| desmoplakin | ○ | | ○ | |
| actin | ○ | | ○ | ○ |
| myosin 9 | ○ | | ○ | ○ |
| Hsp70 | ○ | | ○ | ○ |
| Hspβ-1 | ○ | | ○ | ○ |
| cytokeratin 8 | ○ | | ○ | ○ |
| cytokeratin 9 | ○ | | ○ | ○ |
| cytokeratin 14 | ○ | | ○ | |
| vitronectin | ○ | | ○ | ○ |
| 26S proteasome | ○ | | ○ | |
| non-ATPase | | | | |
| subunit 1 | | | | |
| cathepsin D | ○ | | ○ | ○ |
| retinol-binding | ○ | | ○ | ○ |
| protein-4 | | | | |

LC-MRM of 15 Sets of AH Samples from Patients and their Age- and Sex-Matched Controls

A total of 12 candidate proteins were subjected to LC-MRM assays. It is critical to select unique tryptic peptides for target proteins with good MS signals. We used MRMPilot software (ABSCIEX, Foster City, Calif.) to select multiple tryptic peptides for the given target proteins.

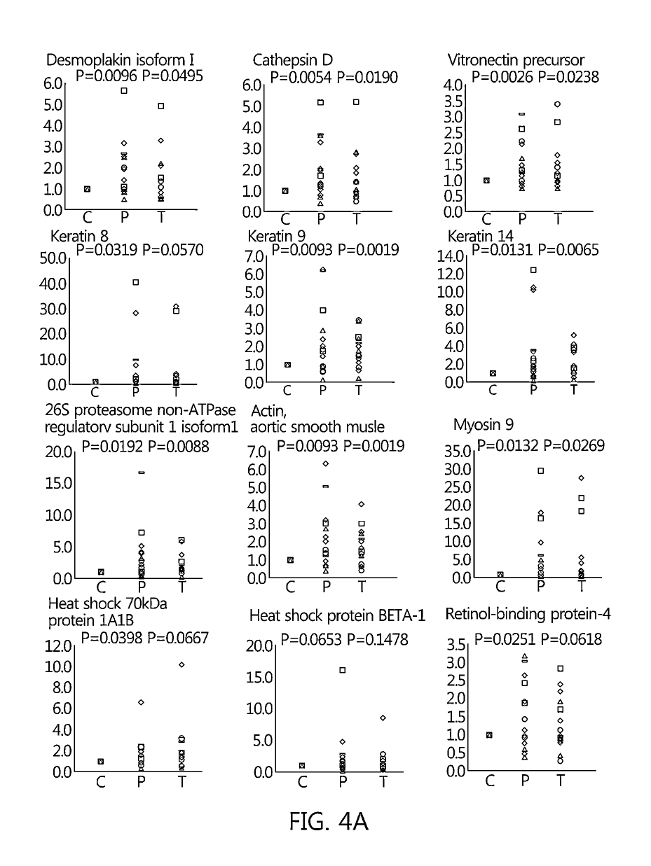

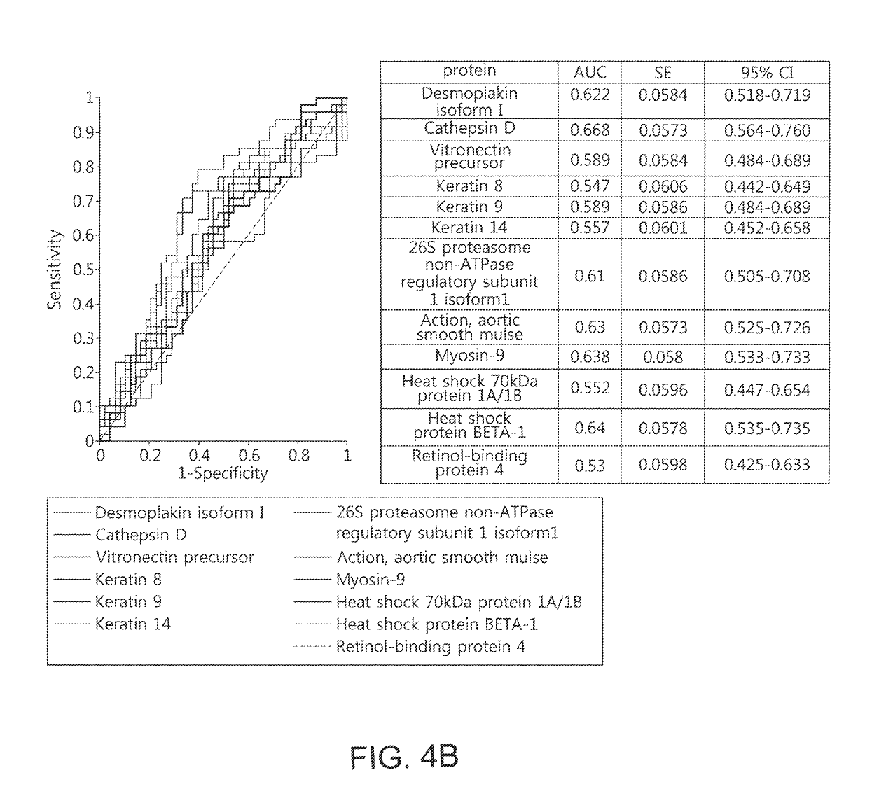

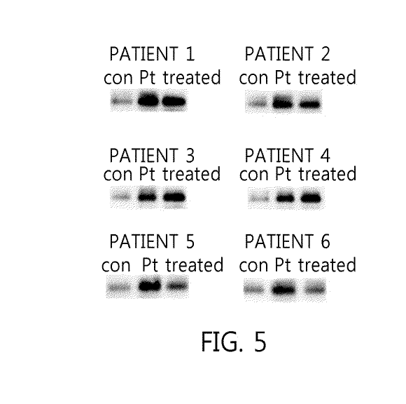

The MRM transitions were optimized for 34 peptides of 12 proteins, as shown in Table 8. The selected peptides were examined by LC-MRM experiments using a QTRAP 5500 triple quadrupole/linear ion trap mass spectrometer. The expression levels of all 12 proteins were elevated in the AH of patients compared to their matched controls (FIG. 4A). Among them, actin (aortic smooth muscle); myosin 9; Hspβ-1; cytokeratins 8, 9, and 14; vitronectin; 26S proteasome non-ATPase subunit 1 (PSMD1); cathepsin D; and retinol-binding protein 4 were upregulated by more than 2-fold compared with the control groups in at least one transition, as determined by t-test analysis. Further evaluation of these proteins as biomarkers was conducted using receiver operating characteristic (ROC) curve analysis, which is widely used in case-control studies. We randomly selected one transition from each of the 12 proteins and performed ROC analysis using all triplicate intensities. The ROC curves show that these proteins can be used to discriminate AMD patients from control subjects. The most notable indicator protein is cathepsin D, with an AUC of 0.668 (FIG. 4B).

In addition to validating the utility of potential biomarkers, including their statistical significance, we sought to determine whether the differential expression of some of these proteins might be associated with responsiveness to ranibizumab for the treatment of AMD. To date, several pharmacogenetic studies of anti-VEGF therapy for neovascular AMD have been published. (2, 55, 56). However, there have been no reports of changes in the secretory proteins in the AH of AMD patients associated with response to anti-VEGF treatment. Although neovascularization (CNV) is known to be triggered by VEGF release, the debate over the precise role of anti-VEGF treatment in neovascular AMD is ongoing (57, 58). Furthermore, despite the undeniable success of anti-VEGF treatment in improving or preserving vision in neovascular AMD patients, its effects seem to be derived mainly from a decrease of sub- and intraretinal edema rather than the regression of CNV itself (59, 60). To identify which secreted proteins were potentially involved in the cellular functions, and thus the development of the anti-VEGF treatment-responsive phenotype, we evaluated central macular thickness (CMT), which reflects sub- and intraretinal edema, using optical coherence tomography (OCT) both before and after treatment and performed statistical analyses to identify correlations between the levels of specific proteins and CMT. Among the 12 proteins analyzed, cytokeratin 8 and Hspβ-1 expression were most closely correlated with better response to treatment, as manifested by a greater decrease in CMT, as measured by OCT, with treatment (Spearman correlation: coefficient 0.56, p-value 0.03 for cytokeratin 8 and coefficient 0.53, p-value 0.04 for Hsp β-1, respectively). Multivariate regression analysis showed that cytokeratin 8 is associated with a greater reduction in CMT (estimate 7.80, error 4.72, p<0.05), underlining the potential utility of this protein in predicting treatment response.

TABLE 8

| | | | | Frag- | | | | |
|---|---|---|---|---|---|---|---|---|
| | Acces- | Protein | Peptide | ment | | | | |
| No. | sion # | Name | sequence | ion | Q1 | Q3 | Dwell | CE |
| 1 | 58530840 | Desmoplakin | VQYDLQK | 2/y5 | 447.24 | 666.35 | 50 | 25 |
| | | isoform I | | | | | | |
| 2 | 4503143 | Cathepsin D | FTSIR | 2/y3 | 312.18 | 375.24 | 50 | 19 |
| | | | FTSIR | 2/y4 | 312.18 | 476.28 | 50 | 19 |
| 3 | 88853069 | Vitronectin | AVRPGYPK | 2/y6 | 444.26 | 717.4 | 50 | 25 |
| | | precursor | DSWEDIFELLFWGR | 3/y6 | 604.96 | 791.46 | 50 | 34 |
| | | | NGSLFAFR | 2/b7 | 456.24 | 737.36 | 50 | 25 |
| 4 | 4504919 | Keratin 8 | FLEQQNK | 2/y4 | 453.74 | 517.27 | 50 | 25 |
| | | | FLEQQNK | 2/b5 | 453.74 | 646.32 | 50 | 25 |
| | | | WSLLQQQK | 2/y5 | 515.79 | 644.37 | 50 | 28 |
| 5 | 55956899 | Keratin 9 | FSSSSGYGGGSSR | 2/y10 | 618.27 | 914.4 | 50 | 32 |
| | | | FSSSSGYGGGSSR | 2/y8 | 618.27 | 740.33 | 50 | 32 |
| | | | FSSSSGYGGGSSR | 2/y9 | 618.27 | 827.36 | 50 | 32 |
| | | | IQDWYDK | 2/y4 | 484.23 | 611.28 | 50 | 26 |
| | | | IQDWYDK | 2/y5 | 484.23 | 726.31 | 50 | 26 |
| | | | IQDWYDK | 2/y6 | 484.23 | 854.37 | 50 | 26 |
| | | | SGGGGGGGLGSGGSIR | 2/y10 | 616.8 | 860.46 | 50 | 32 |
| | | | SGGGGGGGLGSGGSIR | 2/y11 | 616.8 | 917.48 | 50 | 32 |
| | | | SGGGGGGGLGSGGSIR | 2/y8 | 616.8 | 746.42 | 50 | 32 |
| | | | SGGGGGGGLGSGGSIR | 2/y9 | 616.8 | 803.44 | 50 | 32 |
| 6 | 15431310 | Keratin 14 | LAADDFR | 2/y4 | 404.2 | 552.24 | 50 | 23 |
| | | | LAADDFR | 2/y5 | 404.2 | 623.28 | 50 | 23 |
| | | | YETELNLR | 2/y7 | 519.27 | 874.46 | 50 | 28 |
| | | | VLDELTLAR | 2/y5 | 515.3 | 573.37 | 50 | 28 |
| | | | VLDELTLAR | 2/y6 | 515.3 | 702.41 | 50 | 28 |
| | | | VLDELTLAR | 2/y7 | 515.3 | 817.44 | 50 | 28 |
| 7 | 25777600 | 26S proteasome | EPEPNFQLLDNPAR | 2/y10 | 820.41 | 1187.62 | 50 | 41 |
| | | non-ATPase | | | | | | |
| | | regulatory | | | | | | |
| | | subunit isoform1 | | | | | | |
| 8 | 213688375 | Actin, aortic | VAPEEHPTLLT | 4/y5 | 489.77 | 568.35 | 50 | 27 |
| | | smooth musle | EAPLNPK | | | | | |
| 9 | 12667788 | Myosin-9 | YEILTPNSIPK | 2/y6 | 637.85 | 655.38 | 50 | 33 |
| 10 | 194248072 | Heat shock | DAGVIAGLNVLR | 2/y7 | 599.35 | 742.46 | 50 | 31 |
| | | 70kDa | | | | | | |
| | | protein 1A/1B | | | | | | |
| 11 | 4504517 | Heat shock | AQLGGPEAAK | 3/y5 | 314.51 | 515.28 | 50 | 20 |
| | | protein BETA-1 | | | | | | |
| 12 | 55743122 | Retinol-binding | VSSFR | 2/y3 | 298.16 | 409.22 | 50 | 18 |
| | | protein 4 | VSSFR | 2/y4 | 298.16 | 496.25 | 50 | 18 |
| | | | DPNGLPPEAQK | 2/y6 | 583.3 | 669.36 | 50 | 31 |

Table 8. LC-MRM transition chart for the identification of putative protein biomarkers.
Exemplary embodiments of the present invention will now be described in detail with reference to the accompanying drawings.

### Example 1

### Subjects and AH Sample Collection

AH samples were collected at the Department of Ophthalmology, Konkuk University Medical Center, and Seoul, Korea. From Sep. 1, 2011 to Oct. 31, 2012, a total of 26 patients with untreated neovascular AMD and 26 age- and sex-matched patients undergoing cataract surgery (controls) were enrolled in this study. The 26 sets of samples analyzed consisted of samples from controls, samples from patients before treatment (intravitreal injection of ranibizumab), and samples taken from patients at 1 month after the first treatment, for a total of 78 AH samples. The 26 patients were all treatment-naïve; i.e., they had not received any kind of treatment for neovascular AMD prior to their inclusion in the study. Patients with other ophthalmic diseases (e.g., glaucoma, uveitis, or progressive retinal disease), uncontrolled systemic diseases (e.g., uncontrolled diabetes mellitus or arthritis), or who had undergone laser or intraocular surgery were excluded. The control subjects underwent routine senile cataract surgery for visual rehabilitation. AH samples from patients undergoing cataract surgery were used as a control rather than samples from normal eyes for ethical reasons. We matched the ages of the patients (±5 years) with those of the control subjects, and the extent of the cataracts in each individual corresponded to the patient's age. The control subjects did not have any eye disease other than cataracts. The clinical data from the patients and controls are summarized in Table 1. Control samples were obtained immediately before cataract surgery. Samples from neovascular AMD patients were obtained before performing the first intravitreal injection of 0.5 mg ranibizumab and 1 month after the injection (before performing the second intravitreal injection of 0.5 mg ranibizumab). Two sets of samples (6 samples) were used for the whole-protein profiling by LC-ESI-MS/MS analysis (sample set 1 in Table 1). Nine sets of samples (27 samples) were used for the preparation of exosomes from AH, and the subsequent whole-protein profiling was performed by LC-ESI-MS/MS analysis (sample set 2 in Table 1). Because the amount of exosomes per AH sample was small, rather than profiling of each set of samples individually, we performed proteomic profiling of 9 pooled samples each for the controls and for the AMD patients both before and after treatment. Finally, 15 sets of samples (45 samples) were analyzed by LC-MRM (sample set 3 in Table 1), and data were acquired for 15 sets.

For the electron microscopic examination and Western blot analysis of exosomes from AH, 110 AH samples were collected from patients who had neovascular AMD but did not meet the criteria for the experiments described above. These samples were also collected prior to intravitreal anti-VEGF injection. However, these 110 patients had already received some type of treatments for their disease, such as intravitreal injection of ranibizumab or bevacizumab; some patients had other ocular or uncontrolled systemic diseases such as glaucoma or uncontrolled diabetes; and some patients had previously received laser treatments or other intraocular surgeries. The patients ranged in age from 52 to 92 years (average, 71.8±10.3 yrs) and included 62 men and 48 women.

All sample collections and intravitreal injections were performed using standard sterile procedures, and AH samples were obtained by anterior chamber paracentesis using a 30-gauge needle. All surgical procedures were performed by the two retinal surgeons, and no complications were encountered after paracentesis of the anterior chamber. Samples of the AH (100-150 μl) in safe-lock microcentrifuge tubes (1.5 ml) were immediately frozen at −80° C. and stored until analysis. The study followed the guidelines of the Declaration of Helsinki, and informed written consent was obtained from all patients and control subjects. The procedure for AH collection was approved by the Institutional Review Board of Konkuk University Medical Center, Seoul, Korea.

### Example 2

### ARPE-19 Cell Cultures and the Secretome of the Cell Culture Supernatant (CM)

Human retinal pigment epithelial ARPE-19 cells were cultured in DMEM/F-12 supplemented with 10% FBS and 1% antibiotics. Approximately 5×10⁶ cells were plated in each 100-mm culture dish and maintained at 37° C. in a 5% CO₂ incubator to allow proliferation. When cell confluence reached ˜90%, the cells were washed three times in PBS and then treated with 400 μM paraquat (Sigma) at 37° C. for 24 h under serum-free conditions.

At the same time, total cell lysates were prepared from the cells that produced the CM; these lysates were used later in parallel with exosomes for Western blot analyses. A total of 100 ml of cell culture supernatant (CM) was collected and centrifuged at 480×g for 10 min and then at 1,900×g for 10 min to remove dead cells and cell debris. The CM was concentrated to ˜1 ml using an Amicon® Ultracel-10 K molecular weight cut off centrifugal filter device (Millipore) for secretome analysis by LC-ESI-MS/MS.

### Example 3

### The Isolation and Morphologic and Biochemical Characterization of Exosomes from the AH of Patients and CM from ARPE-19 Cell Culture

Exosomes were isolated from the AH of patients and the CM from ARPE-19 cell culture using ExoQuick™ Exosome Precipitation Solution (System Bioscience, SBI) according to the manufacturer's protocol. Briefly, after centrifuging at 3000×g for 15 min to remove cells and cell debris, ExoQuick™ reagent wasa dded to the supernatant and mixed well. Then, the mixture was stored overnight at 4° C. Subsequently, the ExoQuick/sample mixture was centrifuged at 1500×g for 30 min. After centrifugation, the exosomes appeared as a faint yellow-white pellet at the bottom of the vessel. The supernatant was aspirated, and all traces of fluid were removed after the residual ExoQuick™ solution was spun down by centrifugation at 1500×g for 5 min. The exosome pellet was resuspended and subsequently used for transmission electron microscopy (TEM), Western blot analysis, and LC-ESI-MS/MS.

For TEM, exosomes were directly adsorbed onto Formvar-carbon-coated 400 mesh copper EM grids (PELCO®, TEDPELLA Inc.) and dried for 20 min at RT. The specimens were negatively stained with freshly prepared 2.0% aqueous uranylacetate (Fluka), dried, and then photographed using a JEM-1100 transmission electron microscope at an acceleration voltage of 80 kV (JEOL, Japan). Exosomes were defined as relatively homogeneously sized (approximately 50-150 nm in diameter) round membranous vesicles (48) (Thery, C., Amigorena, S., Raposo, G., and Clayton, A. (2006) Isolation and characterization of exosomes from cell culture supernatants and biological fluids. Current protocols in cell biology Chapter 3, 3.22.1-3.22.29).

For Western blot analysis, exosome pellets from the AH of patients and from the CM were resuspended in PBS containing protease inhibitor cocktail (Roche). The protein concentration of the suspension was determined by a modified Bradford Assay (Bio-Rad Laboratories). Cell lysates and exosome sample preparations containing 15 μg protein were loaded per well. The membrane was blocked with 5% nonfat dried milk for 1 h and incubated overnight at 4° C. with the following antibodies: anti-CD63 (Santa Cruz), anti-cathepsin D (Santa Cruz), anti-Hsp70 (BD Sciences), anti-cytokeratin 8 (Abcam), anti-26S proteasome non-ATPase subunit 1 (PSMD1) (LSBio), or anti-Tsg101 (Abcam). Horseradish peroxidase-conjugated goat anti-rabbit or anti-mouse IgG (Cell Signaling) secondary antibodies were used. A chemiluminescence substrate (ECL Prime, Amersham) was used to visualize the immunoreactive proteins.

For immunocytochemistry, ARPE-19 cells were fixed with 4% paraformaldehyde for 1 h at room temperature and then permeabilized with 0.2% Triton X-100 for 10 min. After blocking with 2% bovine serum albumin, the fixed cells were incubated overnight at 4° C. with anti-CD63 (1/1000) antibody. The cultures were then treated with a fluorescence-conjugated secondary antibody (Alexa Fluor 555 anti-rabbit IgG; 1:1000; Invitrogen) for 2 h at room temperature. For negative controls, cultures were treated with the secondary antibody only. The mounted slides were observed under a confocal microscope (FV-1000 Spectral, Olympus) at an excitation wavelength of 568 nm (×800)

### Example 4

### LC-ESI-MS/MS Analysis, Database Searching, and Validation

Trypsin-digested peptides were loaded onto a fused silica microcapillary column (12 cm×75 μm) packed with C18 resin (5 μm, 200 Å). LC separation was conducted under a linear gradient from 3-40% solvent B (0.1% formic acid in 100% ACN) with a flow rate of 250 nl/min for 60 min. The column was directly connected to an LTQ linear ion-trap mass spectrometer (Finnigan, Calif.) equipped with a nano-electrospray ion source. The electrospray voltage was 0.95 kV, and the threshold for switching from MS to MS/MS was 500. The normalized collision energy for MS/MS was 35% of the main radio frequency amplitude (RF), and the duration of activation was 30 ms. The spectra were acquired in data-dependent scan mode. Each full MS scan was followed by MS/MS scans of the five most intense peaks. The repeat peak count for dynamic exclusion was 1, and the repeat duration was 30 s. The dynamic exclusion duration was 180 s, and the width of exclusion mass was ±1.5 Da. The list size of dynamic exclusion was 50. We analyzed the 6 AH samples in triplicate and selected proteins that were identified in at least two replicate analyses for further study (LC-MRM).

The LC-ESI-MS/MS spectra were analyzed using the BioWorksBrowser™ (version Rev.3.3.1 SP1, Thermo Fisher Scientific Inc., CA) with the SEQUEST search engine, which searches the National Center for Biotechnology Information (http://www.ncbi.nlm.nih.gov/) non-redundant human protein database. The search conditions were as follows: trypsin enzyme specificity, no more than two missed cleavages, peptide tolerance of ±2 amu, a mass error of ±1 amu on fragment ions, and fixed modifications of carbamidomethylation on cysteine (+57 Da) and oxidation of methionine (+16 Da) residues. For peptide validation, we applied a decoy database strategy to calculate false-positive (FP) rates utilizing a cross correlation (Xcorr) value. To obtain highly confident data, protein validation with multiple peptide hits was performed for sorted peptides with less than 0.1% FP rates. Thus, we eliminated incorrect (reversed) peptide and protein identifications under the threshold cutoff (error rate<1%, single peptide). For each sample, triplicate experiments were performed. To identify sample-specific proteins, a label-free protein quantification method was applied to triple pairs of protein lists. A sample-specific protein was determined Wits average quantity ratio was at least 1.5 fold.

### Example 5

### LC-MRM

AH samples (each 50 μg) from 15 patients were resolved in 6 M urea and 50 mM ammonium bicarbonate (pH 7.8) in HPLC-grade water. Denatured AH proteins were reduced with 30 mM DTT for 2 h, followed by 1 h of 55 mM iodoacetamide treatment in the dark for alkylation. Alkylated AH samples were digested in solution with sequencing grade modified trypsin (Promega, Madison, Wis.) overnight at 37° C. Formic acid was then added to the sample to stop the digestion. MRM mode was performed on a QTRAP 5500 hybrid triple quadrupole/linear ion trap mass spectrometer (Applied Biosystems/MDS Sciex, Carlsbad, Calif.) equipped with a nanospray ionization source for the quantitative analysis of specific peptides of a protein of interest. A given MRM Q1/Q3 ion value (precursor/fragment ion pair) was monitored to select a specifically targeted peptide corresponding to each candidate protein. The MRM scan was performed in a positive mode with ion spray voltages in the 1800-2100 V range. The MRM mode settings were as follows: curtain gas and spray gas at 10 and 20 psi, respectively, and the collision gas was set to unit resolution. The declustering potential (DP) was set to 100 V. The mass resolution was set to unit using an advanced MS parameter. For the correct LC-MRM, the monitoring of the selected peptide by enhanced product ion (EPI) scan was performed with threshold switching of 100 counts and the selection of rolling collision energy. In positive mode, a product of 30, scan range 100-1000 Da, and two scans were used. In the advanced MS tab, the quadrupole resolution was set to low, the scan speed was 10,000 amu/s, and a dynamic fill time was selected.

Data are expressed as means±S.D. of at least three independent experiments. Statistical comparisons were made using an unpaired two-tailed Student's t-test with confidence level of 95%. The significance level was set at P<0.05.

As set forth above, according to exemplary embodiments of the invention, proteins identified by the present invention can be used for diagnosing, treating and/or monitoring of treatment an age-related macular degeneration.

While the present invention has been shown and described in connection with the exemplary embodiments, it will be apparent to those skilled in the art that modifications and variations can be made without departing from the spirit and scope of the invention as defined by the appended claims.

What is claimed is:

1. A composition for diagnosing an age-related macular degeneration, the composition comprising one or more proteins selected from the group consisting of desmoplakin-I represented by a gene information number (GI No.) 58530840; actin represented by GI No. 213688375; myosin 9 represented by GI No. 12667788; Hsp70 represented by GI No. 194248072; Hspβ-1 represented by GI No. 4504517; cytokeratin 8 represented by GI No. 4504919; cytokeratin 9 represented by GI No. 55956899; cytokeratin 14 represented by GI No. 15431310; vitronectin represented by GI No. 88853069; PSMD1 (26S proteasome non-ATPase subunit 1) represented by GI No. 25777600; cathepsin D represented by GI No. 4503143; a retinol-binding protein 4 represented by GI No. 55743122; and WIF1 (Wnt inhibitory factor 1) represented by GI No. 111125011.

2. A composition for diagnosing an age-related macular degeneration, the composition comprising a protein consisting of desmoplakin-I represented by a gene information number (GI No.) 58530840; actin represented by GI No. 213688375; myosin 9 represented by GI No. 12667788; Hsp70 represented by GI No. 194248072; Hspβ-1 represented by GI No. 4504517; cytokeratin 8 represented by GI No. 4504919; cytokeratin 9 represented by GI No. 55956899; cytokeratin 14 represented by GI No. 15431310; vitronectin represented by GI No. 88853069; PSMD1 (26S proteasome non-ATPase subunit 1) represented by GI No. 25777600; cathepsin D represented by GI No. 4503143; a retinol-binding protein 4 represented by GI No. 55743122; and WIF1 (Wnt inhibitory factor 1) represented by GI No. 111125011.

3. A method for obtaining information about an age-related macular degeneration from a subject, the method comprising:
a) measuring the biomarker in a biological sample obtained from the subject, wherein the biomarker is the protein composition according to claim 2; and
(b) correlating the sample with the age-related macular degeneration when measured value or values is/are higher than that of a normal person.

4. The method of claim 3, wherein for the method, when expression of the protein biomarker in the sample is at least 1.5 time or higher than the expression for the normal person, the sample is correlated to the age-related macular degeneration.
